(19) United States
(12) Patent Application Publication
(10) Pub. No.: US 2011/0015456 A1
(43) Pub. Date: Jan. 20, 2011

(54) GAS TREATMENT APPARATUS-WATER FLOODED SCREW COMPRESSOR

(76) Inventor: John Stephen Broadbent, Auckland (NZ)

Correspondence Address: JACOBSON HOLMAN PLLC, 400 SEVENTH STREET N.W., SUITE 600, WASHINGTON, DC, 20004, US

(21) Appl. No.: 12/736,205

(22) PCT Filed: Feb. 19, 2009
(86) PCT No.: PCT/NZ2009/000021

§ 371 Date: Sep. 20, 2010

(30) Foreign Application Priority Data
Feb. 3, 2008 (NZ) 566845
Oct. 10, 2008 (NZ) 571948

(51) Int. Cl. C07C 9/04; B01D 53/14

(52) U.S. Cl. 585/16; 261/34.1; 95/227; 96/266; 96/265; 95/228; 95/203

(57) ABSTRACT

A gas treatment system for hydrocarbon upgradation comprising a water flooded screw type compressor to receive and discharge water and gas to be treated, a scrubber to receive a gas discharged from the compressors and scrubbing water, a stripper/flasher to receive water and gas discharged from the scrubber and recycle the water for use by one or both compressor and scrubber and a recovery system for the gas from scrubber.

### TECHNICAL FIELD

The present invention relates to gas treatment systems (apparatus and/or processes).

### BACKGROUND ART

We through subsidiary Greenlane Biogas Limited and affiliated Flotech companies support systems for upgrading hydrocarbon gases or biogas. See for example our disclosures in respect of biogas upgrading from raw biogas at www.flotech.com. Particularly see our brochure on our website named Greenlane Brochure Nov06 (A4).pdf. FIG. 1 hereafter is the flow diagram of that brochure.

Commercial scale upgrading biogas to vehicle fuel has been popular in Sweden since 1996. Raw material for biogas production contains approximately 50% sewage sludge; the remainder comes from food industry sludge and organic waste from the surrounding region. The GREENLANE™ system upgrades the raw biogas to deliver clean, dry vehicle fuel (≈97% pure CH4) which is then usually transported via pipeline at 4 bar(g) to the city centre. Biogas fuel is compressed at the city refuelling station and stored at 250 barg, ready for filling cars, buses and trucks.

To upgrade the biogas, the GREENLANE™ Compression/Scrubbing/Flash/Methane Recover system is used, which is an advanced water scrubbing technique with regenerating water system.

GREENLANE™ technology not only produces clean fuel, it does so with an environmentally friendly and safe process: water scrubbing. This technology was developed by Flotech in the early 1990's and has now become the most popular method for production of upgraded biogas, worldwide.

Feedstock of raw biogas is normally supplied to the plant at just above atmospheric pressure and water saturated (RH 100%). Moisture and particulates are removed at the inlet separator then the gas is compressed in two stages up to 9 bar(g) and cooled. Raw gas enters the scrubber at the bottom, contacting the process water in counter-flow towards the cleaned gas exit at the top. The scrubber has especially designed internals, which force the gas to be exposed as much as possible against the process water. CO₂ and H₂S are absorbed into the water, so the gas leaving the scrubber contains 97-98% CH₄ at RH 100%. The gas is then dried in a twin column PSA/TSA drier to control the dewpoint below −80° C. Product gas is analysed; if it does not meet the quality criteria for vehicle fuel, it is recycled back to the compressor inlet for reprocessing.

The process water absorbs some CH₄ during the scrubbing process; this CH₄ is recovered at intermediate pressure in the flashing tank and returned to the compressor to minimise CH₄ losses. The process water then enters the stripping system where the CO, is removed at atmospheric pressure—clean water is pumped from the stripper back into the scrubber process.

The process water is heated (mainly by pump energy input—a two stage pairing of rotary sliding vane type positive displacement compressors). Hence it must be cooled. Where copious cold, fresh water is available, cooling can be achieved by exchanging water from the system, to maintain process temperature. For sites where water is not freely available, or where improved process efficiency is desired, a water chilling system will be installed.

Capacity is water temperature dependent—cold water has greater capacity to absorb CO2 than warmer water. High efficiency is obtained at 7° C. (as shown to the right) by process water chilling. Lowered process temperature gives reduced system pumping costs; hence total energy consumption of a plant with water chilling is lower. With only water exchange for cooling, process temperature will typically be 15° C. or more.

As explained, and as can be seen in FIG. 1, clean biomethane fuel is produced with a reduced use of process water owing to recycling.

It is an object of the present invention to utilise to a synergistic effect a WFS or other water cooled/lubricated type compressor (“WFS type compressor”) in a hydrocarbon gas and/or biogas upgrading system such as that typified by the Greenlane Biogas upgrading process referred to previously.

It is a further or alternative object of the present invention to provide a hydrocarbon and/or biogas upgrading system that utilises water both for cooling and lubricating the compressor system whilst downstream obviating the need for inter-stage cooling and/or liquid separation equipment.

It is a further or alternative object of the present invention to provide a single stage WFS compressor system in a hydrocarbon and/or biogas upgrading system.

It is a further or alternative object to use a WFS type compressor upstream of a water scrubber in a gas treatment system of for example, a kind previous typified.

It is still a further or alternative object to provide a system for gas purification to allow energy usage optimisation.

### DISCLOSURE OF INVENTION

In one aspect the present invention consists in, in a water scrubbing system for upgrading hydrocarbon gases and/or biogas, the use of a water flooded screw (“WFS”) type compressor.

Typifying such WFS type compression systems are those of Svenska Rotor Maskiner AB (“SRM”) such as disclosed in their WO99/11937. That specification itself makes reference to the Swedish equivalent of U.S. Pat. No. 4,758,138. The full content of the aforementioned two specifications is hereby here included by way of reference. Typically such compressors are twin screw.

In another aspect the invention is the use of a WFS type compressor in a compressing/water scrubbing/flashing/recovery (eg, of methane) gas type upgrading system (eg, of a kind typified by, but not restricted to that of the GREENLANE™ CSFR system).

Such WFS type compressors are preferably twin screw.

In another aspect the invention is a gas treatment system, or plant thereof and/or the process thereof, comprising or including

a WFS type compressor to receive and discharge both water and the gas being treated,

a scrubber to receive at least the gas discharge from the compressor (directly or indirectly) and to receive scrubbing water,

a stripper and/or flasher to receive the water and included gas output of the scrubber and to recycle water for use by one or both the compressor and scrubber, and

a recovery system for the gas from the scrubber.

In a further aspect the present invention consists in a gas treatment system, or plant thereof and/or the process thereof, comprising or including

a compressor to receive and compress a gas being treated in an at least substantially oil free environment which includes water,

a scrubber to receive the compressed gas from the compressor and to scrub that gas with water,

a recovery system for the water and at least some of the included gas of the water from the scrubber and to recycle at least part of that recovered water for use by the scrubber and/or compressor, and,

a recovery system for the gas from the scrubber.

Preferably said recovery system for the gas from the scrubber includes a molecular sieve and/or purifying equipment.

Preferably the recovery system for the water and included gas output of the scrubber is adapted to return methane to the scrubber and/or to remove the carbon dioxide and/or remove hydrogen sulphide.

In an aspect the invention is also a process for raising the methane concentration in a methane including gas, said process comprising or including harvesting the gas (optionally with H₂ S removal) after water scrubbing after infeed gas pressurisation in a water cooled and/or lubricated and/or sealed compressor (eg, WFS type, twin screw, or other).

Preferably the process includes water recycling.

In a further aspect the invention consists in a WFS type compressor for or of such a gas treatment system.

In another aspect the invention is gas treated by such a system.

In another aspect the invention is a system substantially of FIG. 2 hereof.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

As used herein the term “and/or” means “and” or “or”, or both.

As used herein the term “(s)” following a noun includes, as might be appropriate, the singular or plural forms of that noun.

### BRIEF DESCRIPTION OF DRAWINGS

Preferred forms will now be described with reference to the accompanying drawings in which

FIG. 1 is as previously defined viz. the flow diagram from Greenlane Brochure Nov06 (A4).pdf found at www.flotech.com, such a flow diagram being that of a commercialised process;

FIG. 2 shows a improved system of the present invention incorporating a WFS type compressor (bounded in a broken line) of a kind as typified in the aforementioned patent specifications, and

FIG. 3 shows (bounded by a similar broken line) the complexities of a system also to be driven by a motor (not shown) that are replaceable when there is the substitution of such a WFS type compressor for those hitherto used and typified in the aforementioned brochure.

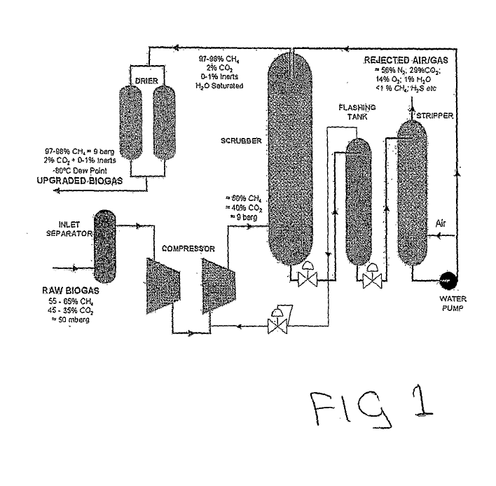

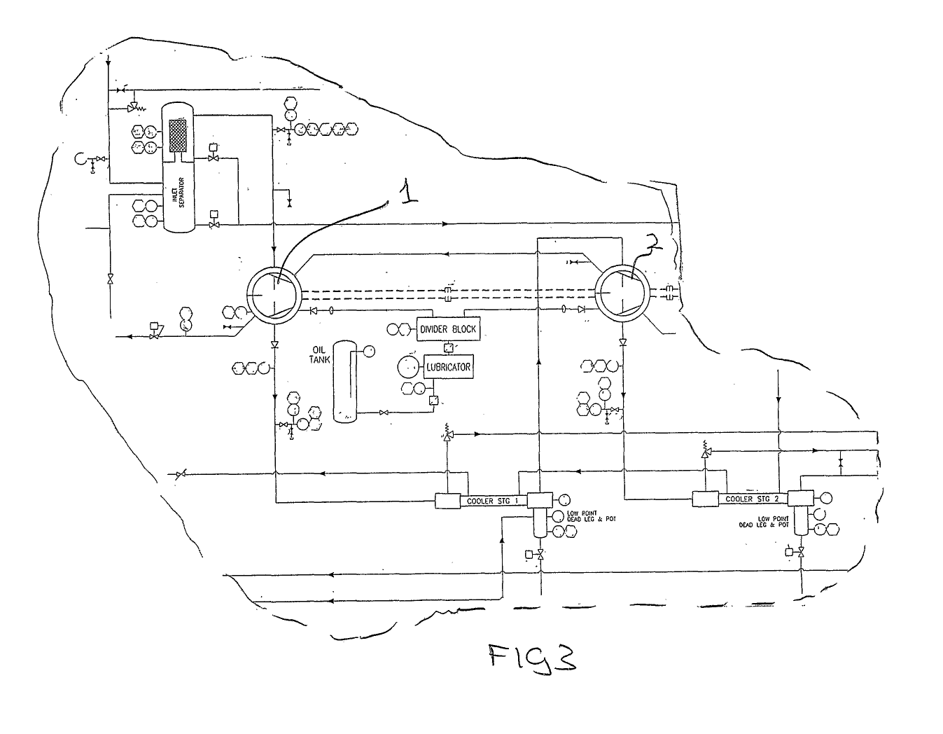

FIG. 1 compressor system is or can be substantially as shown in FIG. 3 where there is a first compressor 1 and a second compressor 2. Shown in FIG. 3 are the complexities of such a flow diagram.

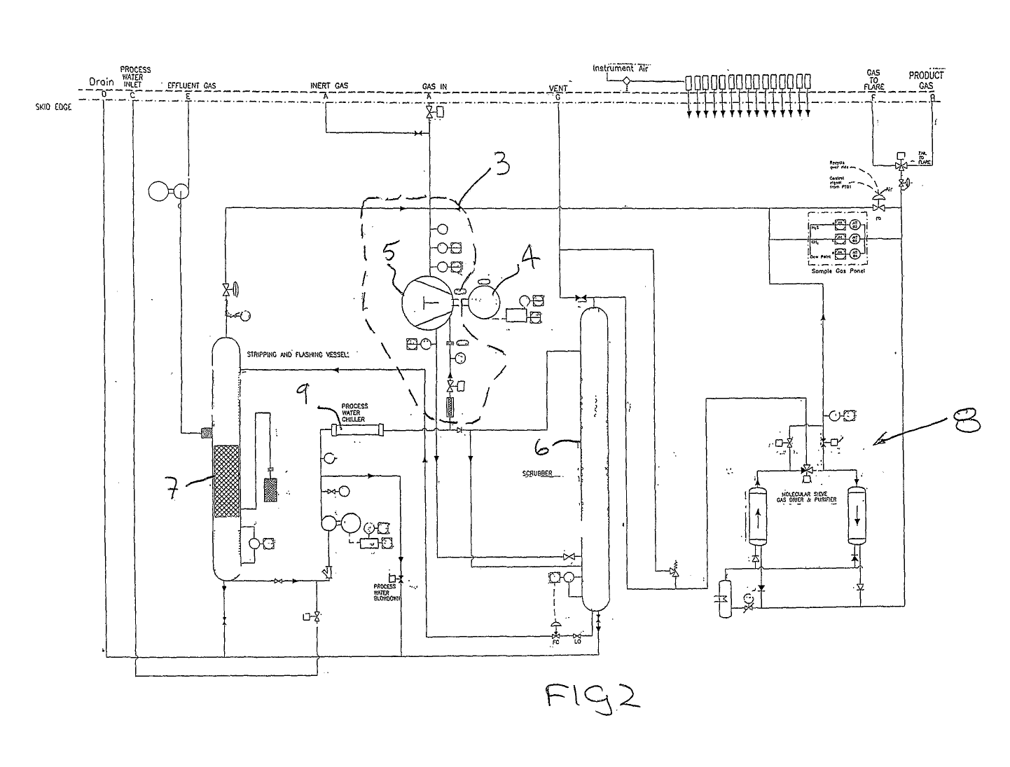

FIG. 2 replaces almost all, or all of the content of FIG. 3 with the enclosed region shown by the broken lines 3. Here a motor 4 operates the WFS twin screw gas compressor 5 in a system that requires little in the way of departure from that shown in FIG. 1. In FIG. 2 there is shown the inflows and/or outflows.

The gas is first compressed by the WFS or twin screw compressor 5 in the presence of water and is then passed for counter flow scrubbing by additional water in the scrubber 6. The water from the scrubber is then passed to the stripping and flashing vessels or vessel 7 from whence there can be recovery and feedback of water to the scrubber.

Shown in FIG. 2 is recovery apparatus 8 which can include a molecular sieve gas dryer and purifier Product gas is that gas high in methane (preferably greater than 97%) that is ducted out as product gas.

Shown in FIG. 2 is a process water chiller 9 that can be interposed between the water recovery of the vessel 7 and the scrubber 6.

Operation can vary depending on through puts, gas types etc. It is envisaged that, for example, the scrubber can operate at, for example, 3 to 15 (preferably about 3 to 7 bar eg, 5 bar) with the compressor having the ability to compress gas to, for example, up to about 15 bar. The molecular sieve and gas purifier can operate at about 3 to 15 bar at a temperature of from 7 to 25° C.

By way of example of water usage in the scrubbing column a useful range is, with respect to a water/inlet gas ratio of from 0.1 to 0.3 (m³ at 20° C./Nm³ per hour).

Persons skilled in the art will appreciate variations that can occur without departure from the present invention and without a denial of any one or more of the benefits of the synergy discussed hereinafter.

It is envisaged in at least one embodiment that, in order to compress a biogas (60% methane, 39% carbon dioxide, 1-2% H₂O/ H₂S/N₂) a twin screw WFS compressor could have a 1 bar inlet pressure to receive at least 30 m³/h of the biogas (eg, up to the thousands).

Intake temperature can be from 10 to 40° C. (eg, 30° C.) and an outlet temperature can be less than 70° C. for a discharge of about 9 bar. Shaft speed(s) of a suitable compressor are 1500 to 6000 rpm.

The wet stream gas from the scrubber can be subject to a H₂S removal process as in our New Zealand Patent Specification 553992 ie, using a single vessel with activated carbon to both dry and then remove H₂S from the wet stream.

Synergy arises in a number of ways.

The drawings in the aforementioned SRM patents shows the rotary screw compressor together with a great deal of equipment that is needed to support the water circulation system.

The Greenlane drawing of FIG. 3 hereof shows the compression system as presently used; this is a two stage compressor with oil lubrication, inter-stage cooling and liquids separation, a gas inlet separator and a great deal of instrumentation that is required to control and provide safety protection for the (old style) compression system. The compression system is shown (without its motor etc).

The drawing of FIG. 2 shows the new combination, whereby the WFS compressor is incorporated within a modified Greenlane process. The WFS compressor and its specific instrumentation etc, is circled in broken lines.

Advantages of the new system:

1. The support system for water treatment normally required for the WFS compressor is already intrinsic in the retained parts of the combined Greenlane system and is thus utilised, eliminating much complexity.

2. The “old” two stage compressor is replaced by the single stage WFS compressor. The reason a single stage WFS compressor can do the job that previously needed two stages is because the cold water injected with the inlet gas on the WFS compressor provides lubrication, sealing and cooling of the compressor internals. It is the cooling benefit that permits a single stage; the injected water cools the gas during the compression process, allowing a much higher compression ratio to be used without over-heating of the compressor.

3. Inter-stage cooling and liquid separation equipment is eliminated by virtue of only requiring a single stage.

4. Cooling of the gas after the single stage WFS compressor is optional, because this can be accomplished by the injected water.

5. No lubrication oil is required, which avoids contamination of the water scrubbing system and saves the cost of oil, which was previously a total loss system and thus wasted. Costs of contaminated oil disposal are avoided.

6. Gas inlet separation is not required for the WFS compressor. On the “old” system this was needed to remove water mist and droplets that are naturally present in the water-saturated biogas, to prevent damage to the oil lubricated compressor. As the WFS compressor is water lubricated (and cooled) the water delivered with the biogas is beneficial.

7. Liquid separation of the gas after compression is not required because the water is separated and re-processed by the downstream system.

8. Partial scrubbing of the biogas will take place during compression due to the agitated close contact with the cold water that is injected into the WFS compressor. This reduces the required size and/or increases the available capacity of the new process system.

The water flooded/lubricated screw compressor (WFS) offers significant advantages over conventional oil fed rotary vane compressors in this application because the water fed to the compressor for sealing and lubrication (10-30% of recycled water flow and chilled to 0.5-5° C.) is further used as process water in the scrubber to upgrade the raw biogas.

Furthermore using WFS technology eliminates the need for two stage compression and inter-stage cooling. Cooling water temperature rise across the WFS compressor is typically between 2-10° C. with gas discharge temperatures anywhere between 3-15° C. Gas discharge temperatures from a two stage rotary vane compressor and after final cooling are typically in the range 50-60° C.

Cooling water is normally injected into the compressor at a pressure between 65-100% of the compressor discharge pressure.

1. A water scrubbing system for upgrading hydrocarbon gases and/or biogas, the use of a water flooded screw (“WFS”) type compressor to substantially scrub the hydrocarbon gases and/or biogas and to feed the resultant scrubbed gases or biogas for downstream processing.

2. A use of a water flooded screw (“WFS”) type compressor in a compressing/water scrubbing/flashing/recovery gas type upgrading system to substantially scrub the gas to be upgraded.

3. The use of claim 2 wherein said WFS type compressor is twin screw.

4. A gas treatment system, or plant thereof and/or the process thereof, comprising or including a water flooded screw (“WFS”) type compressor to receive the gas to be treated, substantially scrub that gas and discharge both water and the scrubbed gas being treated, a secondary scrubber to receive the scrubbed gas discharge from the compressor and to receive scrubbing water,
A stripper and/or flasher to receive the water and included gas output of the scrubber and to recycle water for use by one or both the compressor and scrubber, and a recovery system for the scrubbed gas from the scrubber.

5. A gas treatment system comrprising or including a compressor to receive and compress a gas being treated in an at least substantially oil free environment which includes water, a scrubber to receive the compressed gas from the compressor and to scrub that gas with water, a recovery system for the water and at least some of the included gas of the water from the scrubber and to recycle at least part of that recovered water for use by the scrubber and/or compressor, and, a recovery system for the gas from the scrubber.

6. The system of claims 5 wherein said recovery system for the gas from the scrubber includes a molecular sieve and/or purifying equipment.

7. The system of claim 5 wherein the recovery system for the water and included gas output of the scrubber is adapted to return methane to the scrubber and/or to remove the carbon dioxide and/or remove hydrogen sulphide.

8. A process for raising the methane concentration in a methane including gas, said process comprising or including harvesting the gas after water scrubbing after infeed gas pressurisation in a water cooled and/or lubricated and/or sealed compressor.

9. The process of claim 8 wherein the compressor is of a water flooded screw type.

10. The process of claim 8 which includes water recycling.

11. The water flooded screw type compressor of a gas treatment system of claim 5.

12. The gas treated by a system of claim 5.

13. (canceled)
